(19) United States
(12) Patent Application Publication
(10) Pub. No.: US 2006/0270908 A1
(43) Pub. Date: Nov. 30, 2006

(54) Self Inserting Intraolcular Light

(76) Inventors: K. Peter Luloh, Stuart, FL (US); Michael Annen, Fort Pierce, FL (US); Paul Tornambe, Poway, CA (US)

Correspondence Address: BEUSSE WOLTER SANKS MORA & MAIRE, P. A., 390 NORTH ORANGE AVENUE, SUITE 2500, ORLANDO, FL, 32801, US

(21) Appl. No.: 11/383,808

(22) Filed: May 17, 2006

Related U.S. Application Data
(60) Provisional application No. 60/682,281, filed on May 18, 2005.

(51) Int. Cl. A61B 1/06

(52) U.S. Cl. 600/182

(57) ABSTRACT

An apparatus and method for illuminating a region of an eye is provided and may include at least one optical fiber in fluid connection with a light source for allowing light to be transmitted to a probe for diffusing light into the region of the eye. The probe be formed from a portion of the at least one optical fiber and may be directly inserted into a portion of the eye so that light is diffused into the eye to illuminate the region. The probe may be secured in a substantially fixed position when the probe is inserted within a portion of the eye such as by a frictional engagement between the diffusing means and the eye. A plurality of probes may be inserted within the eye where each probe may include a substantially tapered distal end to facilitate insertion into the eye. The distal end of each probe is formed with a cutting device to enable the probe to be pressed into the eye with the cutting device forming an aperture through which the optical fiber protrudes.

### CROSS REFERENCE TO RELATED APPLICATIONS

This application claims priority to a provisional application filed on May 18, 2005 having application No. 60/682,281, the specification of which is incorporated herein by reference.

### BACKGROUND OF THE INVENTION

This invention relates in general to eye surgery and more particularly to a method and system for internally lighting or illuminating an eyeball to enable a surgeon to perform various surgical procedures on the eye and allow the use of varying instruments during these procedures.

A common surgical procedure performed on eyes, for example, is pars plana vitrectomy. This procedure is a closed vitreous surgical technique for operating on the eye wherein the surgical field is observed through the pupil and instrumentation is inserted into the vitreous cavity through surgical cuts or sclerotomies. These cuts may be fitted with ports to prevent leakage of intraocular fluid during the procedure. Visualization is accomplished using a viewing system, such as a binocular indirect opthalmomicroscope system disclosed in U.S. Pat. No. 4,710,000 or 5,009,487. Intraocular pressure is regulated by infusion of fluid through a separate sclerotomy port. Illumination of the back of the eye or fundus may be originated from an external source through the pupil, or internally through fiber optics. It has been generally recognized that internal illumination with fiber optics is superior to external illumination and is not as dependent on variances in pupillary dilatation or clarity of the ocular media. A frequent practice is to employ a three- or four-port procedure, utilizing one or two ports for exchangeable working instruments, another port for infusion, and another port for illumination using a source such as a ceiling light available from D.O.R.C. Company, Geervleit, The Netherlands, or a chandelier system available from Grieshaber, Schafthausen, Switzerland.

It is known to incorporate optical fibers into the working end of the surgical instrument. This eliminates the need for a separate illumination port and offers the advantage of directing the light beam together with the instrument onto the target site. Instrument sizes must, however, be correspondingly increased and larger scierotomies may be necessary. An alternative procedure is to employ an illuminated infusion cannula to integrate the infusion and illumination functions at a single point.

One example of a combined infusion cannula and illumination source is disclosed in U.S. Pat. No. 4,820,264. The '264 device comprises an infusion channel through which light transmitting fibers are passed for directing light into the eyeball at the point of discharge of the intraocular irrigating solution. Such illumination is not automatically directed by manipulation of the cutting instruments. Moreover, the fibers are run directly within the infusion channel, and illumination and infusion portions are non-separable near the eye. This may cause a less than optimal illumination of the area or field of view undergoing the surgical procedure.

The integrated lighting concept has been extended to provide illuminated cannulas at multiple ports having channels through which either infusion fluids or surgical instruments can be passed. Such a multiport illuminated cannula system is disclosed in U.S. Pat. No. 5,632,740. Such multiport illuminated cannula may comprise a plurality of light transmitting fibers annularly arranged about a central instrument-receiving working channel. Such device has the advantage that fibers are located external to the working channel. The channels may be, however, awkward to seal upon instrument removal and, if used for infusion purposes, may lack expedient infusion tube interfaces and, as in the '264 device, discharge fluid directly at the optical fiber terminations, thereby interfering with illumination.

A general problem with the above-described devices is that they are complicated lighting systems and the channel for inserting instruments to the eye is inseparable from channels allowing for the illuminating sources to direct light to the surgical area or field of view. This combination may limit the surgical diversity of a device and/or create the need to move the device around during surgery to properly illuminate the surgical area or target. This need may result because the optical fibers directing light for illuminating the field of view concentrate the light too directly on a specific area or areas. Light directed in this manner may not be sufficiently diffused for continuously and uniformly illuminating a field of view. Furthermore, other illumination devices used during eye surgery, such as a common light pipe, may require that a surgeon hold or guide them in one hand during surgery. This type of mono-handed surgery may limit the range of a surgeon's procedures or impede the surgical precision necessary for performing eye surgery.

### SUMMARY OF THE INVENTION

In view of the above, it would be advantageous to provide an apparatus for illuminating a field of view during a procedure on an eye that delivers diffused light to the eye so that manipulation of the light source is not required during the procedure. This allows for a surgeon to perform the procedure without interruption for redirecting light and provides optimal illumination of the field of view. It would be further advantageous to provide such a light source as an independent instrument that does not need to be held in one hand during surgery once in position. This allows for a surgeon to use both hands during surgery to use and manipulate various surgical instruments without impairing the illumination of the field of view.

An apparatus for illuminating a region of an eye is provided that may include at least one optical fiber in fluid communication with a light source for providing light to a means for diffusing light within an eye. The means for diffusing light into the region of the eye may be connected with a distal end of the at least one optical fiber. A means for securing the diffusing means in a substantially fixed position when the diffusing means is inserted within a portion of the eye is also provided. In one exemplary embodiment, the diffusing means may be a substantially cylindrical probe made of a transparent or translucent material for allowing light to be admitted and diffused in the region of interest when inserted within the eye. The diffusing means may be secured in a substantially fixed position when inserted into the eye. In one exemplary embodiment the diffusing means may be held in place by a friction engagement established between an outer layer or surface of the diffusing means and an interior portion of the eye when the diffusing means is inserted into the eye.

One aspect allows for providing an apparatus for illuminating at least one field of view of an eye during surgery on the eye that may include a light source and means for transmitting light from the light source to a plurality of light emitting probes. At least one of the probes may be inserted within the eye such that an outer layer or surface of the at least one probe contacts an interior portion of the eye while inserted. One embodiment allows for a means for securing the probe to include a friction engagement established between the outer layer of a probe and a portion of an interior surface of the eye when the probe is inserted in the eye. Alternate securing means may include a hooked portion or bend in the transmitting means, such as an optical fiber, that is proximate a base of the at least one probe. The hooked portion may be configured in relation to the at least one probe such that a stress is created at an angle to a longitudinal axis of the at least one probe when inserted into the eye. In this respect, the stress created prevents the at least one probe from being unintentionally moved or pulled out of the eye and positions the optical fiber so it does not interfere with a surgeon while performing the surgery.

Another aspect allows for a method for illuminating a field of view within an eye, the method including inserting at least one light-diffusing probe directly into an eye such that the at least one probe extends into an interior portion of the eye and diffuses light into the eye to illuminate the field of view. The at least one probe may be inserted into the eye to a depth such that a distal end of the probe does not protrude into the field of view. The method may include securing the at least one probe in a substantially fixed position while inserted within the eye. A plurality of probes may be inserted proximate a peripheral edge of the eye for providing a uniform and continuous illumination of a field of view.

A further aspect of the invention resides in both a method and apparatus for inserting the probe into an eye of a patient without having to create a pre-formed aperture into the eye before inserting the probe. This aspect of the invention addresses the issue of both creating the aperture such as by inserting a hypodermic needle into the eye to create the aperture and subsequently locating the preformed aperture for insertion of the probe. Since it is desirable to make the aperture as small as possible in order to minimize any drainage from the eye, the aperture is often hard to see for insertion of the probe. In this aspect of the invention, the probe is incorporated into a cutting device that creates the aperture into the eye concurrently with insertion of the probe.

### BRIEF DESCRIPTION OF THE DRAWINGS

FIG. 1 is a plan view of an exemplary embodiment of an apparatus for providing lighting via a fiber optic link to an interior surgical site;

FIGS. 2A and 2B are sectional views of a probe assembly in one form of the invention;

FIGS. 3A and 3B are side and top views respectively of a tool for use with the probe of FIG. 2; and

FIGS. 4A and 4B illustrate use of the probe of FIG. 2A and the tool of FIG. 3A.

### DETAILED DESCRIPTION OF THE INVENTION

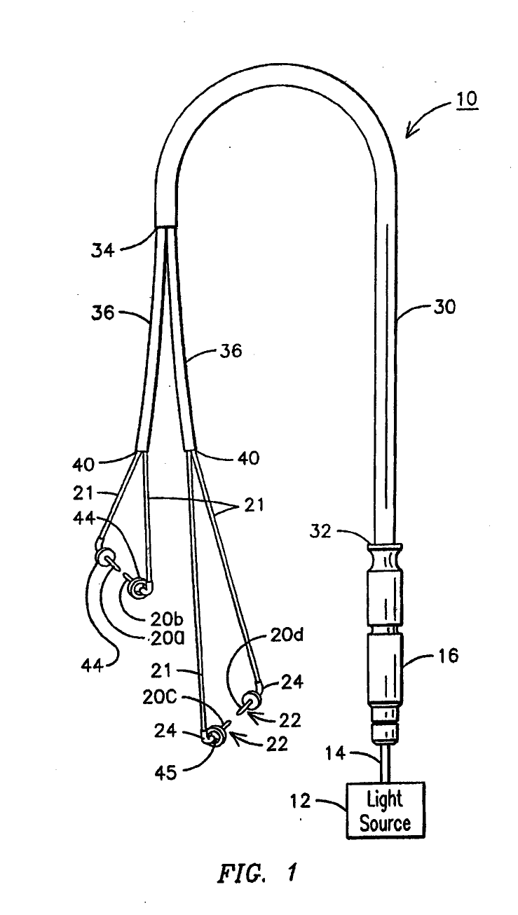

FIG. 1 illustrates an exemplary embodiment of an apparatus 10 for providing light via a fiber optic link to a surgical location such as an interior of an eye. The apparatus 10 may receive light transmitted from a conventional light source 12 connected via a light-transmitting conduit 14 to a connector 16. The connector 16 may be constructed of aluminum for example and may be fitted with suitable connectors (not shown) for connecting a plurality of commercially available optical fibers 20, such as 25 gauge fibers, with the light source 12. The length of each optical fiber 20 may be encased within an opaque outer layer 21 that may be a black polyvinyl chloride (“PVC”) such as a heat shrinkable PVC material, for example. The outer layer 21 may be applied to prevent light from escaping the optical fibers 20 during transmission.

The plurality of optical fibers 20 may function as a means for transmitting light to a plurality of light-emitting probes 22 disposed on a distal end 24 of the plurality of optical fibers 20. Each light-emitting probe 22 comprises an assembly of components through which a continuous portion of a respective one of the plurality of optical fibers 20 protrudes in the operative position. In this respect, the outer layer 21 may cover an optical fiber strand 20 so that a portion of the distal end 24 of the strand is exposed to function as the light-emitting probe 22. The optical fibers 20 may be selected having properties that will permit the length of probe 22 to function as a light diffusing means, such as a translucent optical fiber glass for example. Alternately, the optical fibers 20 may be selected having properties that will minimize light diffusion along the length of probe 22 so that the light is transmitted to the end of the probe 22 where it may be diffused into a field of view within the eye as more fully described below. A sheath or tubing 30 may be provided and encase the plurality of optical fibers 20 to protect them from damage, contain light within the fibers and maintain them in a bundle for ease of handling and connection to the connector 16. The tubing 30 may be fabricated of conventional flexible material such as black silicon tubing, for example. The tubing 30 may extend from an upper end 32 of the connector 16 to a dividing point 34 where the plurality of optical fibers 20 may split into discrete groups. For example, the apparatus 10 as shown allows for four optical fibers 20 to be bundled within the tubing 30 then split into two groups of two at the dividing point 34 where each group may be encased by a sheath or tubing 36. The tubing 36 may be fabricated of conventional flexible material such as black PVC tubing, for example. The tubing 36 may extend from the dividing point 34 to an exiting point 40 where the optical fibers 20 may no longer be encased by tubing 36. This allows for each of the optical fibers 20 to be manipulated as a single optical fiber strand for ease of a surgeon's placement within an eye. Each optical fiber 20 may emerge from the tubing 36 at exit point 40 with or without the opaque outer layer 21 as a function of surgical specifications, for example. The tubing 30 and 36 provide protection to the optical fibers 20 and help to contain light within the fibers. They also provide a convenient way to control the bundled fibers and place the light-emitting-probes 22 for a surgical procedure. Bundling a portion of the fibers also helps to ensure that the fibers 20 do not interfere with a surgeon or technician during surgery. For example, the light source 12 may be located several feet away from the point at which the probes 22 are being used during surgery. Tubing 30 and 36 allow for the single fiber optic strands 20 to be bundled together up to the point where a surgeon needs each strand to be flexible and individually manipulated. Alternate embodiments allow for tubing 30 to extend to the exiting point 40 in which case each of the plurality of optical fibers 20 would exit tubing 30 as individual strands. Another alternate embodiment allows for each optical fiber 20 to exit from the connector 16 as an individual strand. Other configurations for bundling or controlling the plurality of optical fibers 20 will be apparent to those skilled in the art.

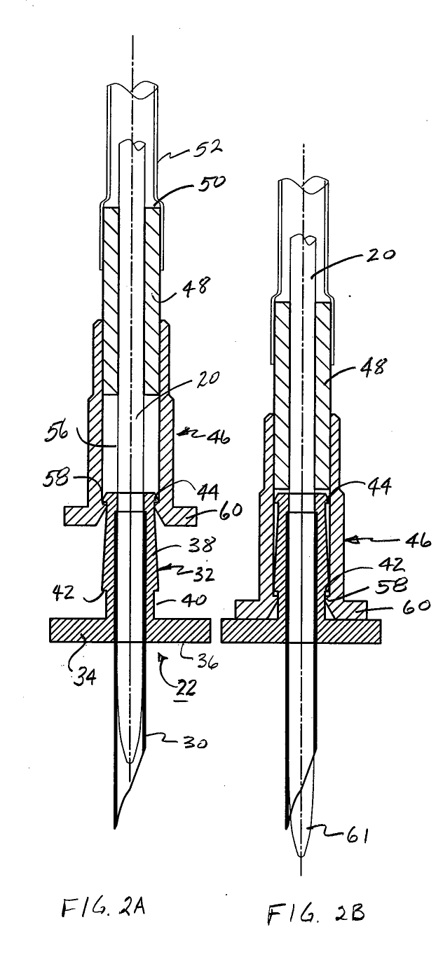

Turning now to FIG. 2A, there is shown a cross-sectional view of one of the light-emitting probes 22. As noted above, each probe 22 is an assembly of components with an exposed portion of the optical fibers 20 extending therethrough. An exposed portion of a fiber 20 extends into a hollow cutting device 30 that is attached to a support member 32. The device 30 may be an end portion of a hypodermic needle of suitable small outer diameter to be inserted into the eye without creating an aperture that will result in fluid leakage from the eye. The support member 32 may be bonded to the device 30 or the device 30 may be molded into the support member during formation thereof if the support member is a molded plastic material. The support member 32 has a footplate 34 with a surface 36 adapted for resting on a surface of an eye when the device 30 is in an inserted position in an eye. The support member 32 further comprises a tubular member 38 extending oppositely from surface 36 of plate 34. The tubular member 38 includes a reduced diameter section 40 adjacent the plate 34 that defines a first flange 42. Extending from flange 42, the tubular member 38 tapers slightly up to a second circumferential flange 44 at an upper end of the member 38.

The support member 32 is configured and designed to telescope into an outer tubular member 46. The member 46 is fixed in position with respect to optical fiber 20. One arrangement for fixing member 46 to fiber 20 is to use a sleeve 48 through which the fiber passes with the sleeve being bonded to the fiber by a typical adhesive such as Loctite 4981. The sleeve 48 is preferably opaque to optical frequencies and the joint formed between an end of the sleeve at 50 where the sleeve abuts the fiber coating 54 may be covered by tubing 52 such as heat shrinkable tubing. It will be appreciated that the sleeve 48 could be formed integrally with member 46 rather than being a separate part since the purpose is to provide connection to fiber 20 while creating a larger inner diameter area at 56 for receiving the tubular member 38. The member 38 is retained in the area 56 by a flange 58 circumscribing a lower entry into the area 56. An outer flange 60 circumscribes the outer lower portion of the entry into area 56 to provide additional support for the member 46.

In FIG. 2b, the tubular member 38 is shown telescoped into the member 46 so that an end 61 of the optical fiber 20 extends outward of the needle or device 30. The member 38 is held in this retracted position by means of the lower flange 42 engaging the flange 58. The footplate 34 abuts against the eye surface and controls the depth of insertion of the needle into the eye in accordance with a pre-selected depth established by the portion of the needle or device 30 extending out of the member 32. During the initial insertion of the needle into the eye, the optical fiber indicated at 20 is recessed into the device 30 so that the fiber end does not protrude beyond the sharpened end 62 of the needle. Once the footplate 34 with the attached device 30 has been pushed into position against the eye so that the device is inserted through the eye wall, the member 38 is then pushed into the member 46 so that the flange 42 engages the flange 58 to hold the member 38 in a retracted position. The optical fiber 20 is fixed to the member 46 so that moving the member 46 towards member 32 causes the end of the optical fiber 20 to extend outward of the device 30 whereby light can be directed into the eye.

The device 30 may be configured as a needle portion and be provided with various types of sharpened cutting surfaces including a conventional hypodermic needle cut or a circular cut similar to that used in artery punches. For example, FIG. 2A shows a typical sharpened end similar to that of a hypodermic needle.

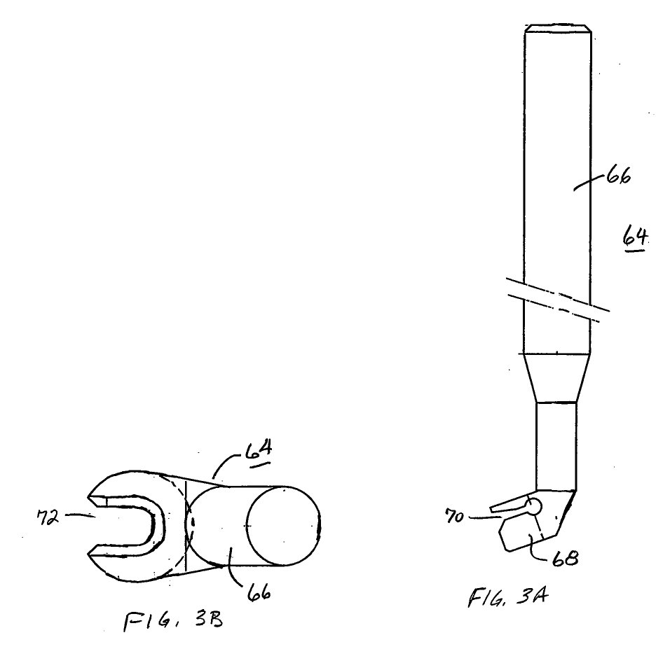

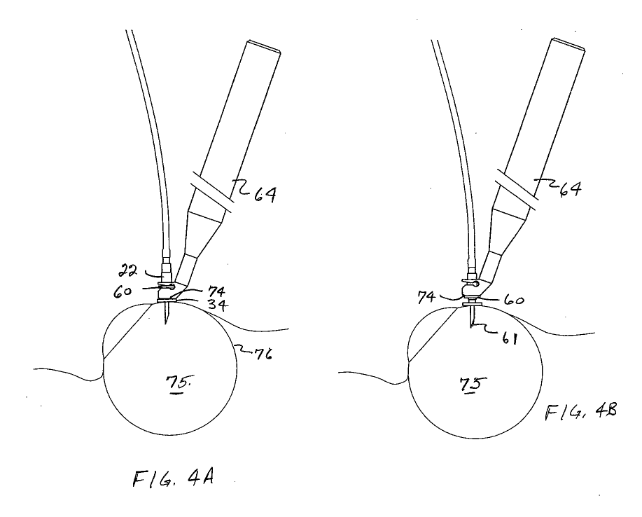

Turning now to FIGS. 3A and 3B, there are shown a side view and a top plan view of a tool 64 that is adapted specifically for use with the probes 22 to enable easy insertion of the probe into an eye. The tool 64 includes an elongate handle 66 that tapers down to a holder 68. The holder 68 includes a slot 70 that is sized to fit onto the outer flange 60 of member 46. The holder 68 also includes a slot 72 transverse to slot 70 that creates a fork-like end so that the member 38 or the member 46 can be inserted into the holder 68. Referring to FIGS. 4A and 4B, the use of the tool 64 in inserting the probe 22 into an eye 75 is illustrated. In FIG. 4A, the probe 22 is associated with the tool 64 such that the flange 60 is inserted in slot 70 and a base 74 of the tool engages an upper surface of footplate 34 so that the probe can be pushed downward and allow the device 30 to penetrate the eye wall 76. The tool 64 is then withdrawn from the probe 22 and re-associated with the probe such that the base 74 is engaged against an upper surface of the flange 60 as shown in FIG. 4B. When the tool 64 is now pushed toward the eye 72, the member 46 moves with respect to member 32 causing the extension or tubular member 38 to be retracted into member 46 as member 46 is moved towards the eye. As a result, the end 61 of the optical fiber 20 extends outwardly of the needle 30 to provide an exposed light source within the eye 75. The tool 64 is then removed from the work area around the eye.

While exemplary embodiments in accordance with aspects of the present invention have been shown and described by way of example only, numerous variations, changes and substitutions will occur to those of skill in the art without departing from the invention herein. Accordingly, it is intended that the invention be limited only by the spirit and scope of the appended claims.

We claim:

1. An apparatus for illuminating a region of an eye, the apparatus comprising:
at least one optical fiber coupled to a light source;
a probe coupled to an end of the at least one optical fiber and adapted to be inserted into an eye for illuminating the region with light from the optical fiber, the probe including a cutting device for enabling concurrent insertion of the probe and the end of the optical fiber into the eye.

2. The apparatus of claim 1 wherein:
the cutting device is releasably engaged with the end of the optical fiber and the fiber is retracted within the cutting device until the device is inserted into the eye.

3. The apparatus of claim 2, wherein the cutting device is held in secured arrangement with eye by a friction engagement established between the outer surface of the cutting device and a wall portion of the eye.

4. The apparatus of claim 2, wherein the probe comprises a first member to which the cutting device is attached and a second member for telescopically receiving at least a portion of the first member such that the first member can be telescoped into the second member to expose the end of the optical fiber.

5. The apparatus of claim 4, wherein the first member comprises a footplate for engaging a surface of the eye for establishing a preselected depth of insertion of the cutting device into the eye.

6. The apparatus of claim 5, wherein the first member comprises a tubular extension from the footplate towards the second member, the second member including an aperture for receiving the tubular extension to allow the first member to telescope into the second member.

7. The apparatus of claim 6, wherein the second member is fixed to the optical fiber such that telescoping the first member into the second member is accomplished by pushing the second member onto the first member and advancing the optical fiber through the first member.

8. The apparatus of claim 7, wherein the second member includes a circumferential flange for engaging the footplate of the first member when the members are in their telescoped relationship, and including a hand tool for simultaneously engaging the flange and the footplate for pressing the probe into insertion into an eye.
